(19) United States
(12) Patent Application Publication
(10) Pub. No.: US 2022/0143157 A1
(43) Pub. Date: May 12, 2022

(54) NOVEL USES OF BOTULINUM NEUROTOXIN FOR THE TREATMENT OF TREMOR

(71) Applicant: Merz Pharma GmbH & Co. KGaA, Frankfurt am Main (DE)

(72) Inventors: Janos CSIKOS, Frankfurt am Main (DE); Irena PULTE, Frankfurt am Main (DE); Reinhard HIERSEMENZEL, Frankfurt am Main (DE); Michael ALTHAUS, Frankfurt am Main (DE); Christiane NALASKOWSKI, Frankfurt am Main (DE); David SIMPSON, New York, NY (US); Bahman JABBARI, Newport Coast, CA (US); Mandar JOG, London (CA); Jack LEE, London (CA)

(21) Appl. No.: 17/431,997

(22) PCT Filed: Feb. 18, 2020
(86) PCT No.: PCT/EP2020/054201

§ 371 Date: Aug. 18, 2021

(30) Foreign Application Priority Data
Feb. 21, 2019 (EP) 19158541.3

(51) Int. Cl. A61K 38/48; A61K 9/00; A61P 25/14

(52) U.S. Cl. CPC A61K 38/4893; A61P 25/14; A61K 9/0019

(57) ABSTRACT

This invention relates to novel uses of botulinum neurotoxin in treating a tremor, in particular to a botulinum neurotoxin for use in treating a tremor of the upper limb comprising the administration of a botulinum neurotoxin to at least one muscle of the forearm/wrist, elbow and shoulder, wherein the botulinum neurotoxin is administered to at least one muscle of the forearm/wrist selected from the group of M. extensor carpi ulnaris (ECU), M. extensor carpi radialis (ECR), M. pronator quadratus (PQ) and M. supinator in a dosage in the range of 2 to 6 U and wherein the botulinum neurotoxin is administered to at least one muscle of the elbow in a dosage of about 20 U and to at least one muscle of the shoulder in a dosage of about 15 U.

### FIELD OF THE INVENTION

This invention relates to novel uses of botulinum neurotoxins for the treatment of a tremor, in particular uses of botulinum neurotoxin in treating upper limb tremor in adults or children with essential tremor or for any other reason where reduction of tremor provides a benefit for a subject.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TxNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin, which is transcytosed into central neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/G with further subtypes. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TxNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TxNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as the treatment of fine wrinkles. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

Clostridial neurotoxins display variable durations of action that are serotype specific. The clinical therapeutic effect of BoNT/A lasts approximately 3 months for neuromuscular disorders and 6 to 12 months for hyperhidrosis. The effects of BoNT/E, on the other hand, last about 4 weeks. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

The longer lasting therapeutic effect of BoNT/A makes it preferable for certain clinical uses and in particular for certain cosmetic uses compared to the other serotypes, for example serotypes B, C, D, E, F, G.

Tremor is the most prevalent movement disorder. Its unmet medical need is represented by a wide variety of clinical conditions which result in the symptom of tremor of different body parts. These symptoms include e.g. jerky, rhythmical movements of head, arms, hands, fingers, legs, feet, trunk, vocal cords or involuntarily additional movements thereof. As a result, functional disability of the affected body part and reduction in the quality of life in subjects occur. Per definition, tremor is an oscillatory involuntary movement of muscles, which can occur in rest or in action. Tremor could affect posture of the body part during voluntary movement (kinetic tremor) depending on the movement. The respective underlying diseases and tremor symptoms and syndromes are not clearly differentiated by clinical phenomenology. A consensus on tremor syndromes were recently delineated (Bhatia et al. Movement Disorders, Vol. 33, No. 1, 2018), but consistent use of the suggested terminology is not implemented yet. Due to the unclear cause in some instance (e.g. Essential tremor), and pathomechanism of the underlying conditions this consensus is expected to necessitate modifications in the future.

Causes of tremor can be various and can be related to physiological functions (cold induced physiologic tremor), pathological origin (dyskinetic tremor), Parkinson's tremor, Holmes tremor, essential tremor, drug-related side effects, alcohol- or drug-withdrawal symptoms (delirium tremens) or functional tremor (also called psychogenic tremor). The coexistence of those factors renders a clear distinction of causes challenging. The descriptors like diagnoses or symptoms as listed above are used in accordance with the Task Force on Tremor of the International Parkinson and Movement Disorder Society (IPMDS) clinical diagnostic criteria [Bhatia et al. Movement Disorders, Vol. 33, No. 1, 2018] and earlier published consensus statements.

Essential tremor (ET) is the most prevalent adult movement disorder [Hess et al. Tremor Other Hyperkinet Mov 2012; 2:], [Jankovic et al. Movement Disorders Vol. 11, No. 3, 1996, pp. 250-256]. Diagnosis of ET is coded in ICD-10 (2018) with G250 (G25.0), uniquely describing only this medical entity. The most recent prevalence estimate [Louis et al. Tremor and Other Hyperkinetic Movements 2014] calculates approximately 7 million US citizens with ET (including hand, head, and palatal manifestations). Existing therapies have several shortcomings and do not sufficiently treat the entire patient population. Oral medications (propranolol, the only FDA approved drug for ET, and primidone, off label) have been used since the 1970's but with insufficient response rates. In addition, these treatments are associated with a high rate of systemic adverse events (e.g. hypotension, sedation, nausea, ataxia, or confusion). Beta blockers like propranolol have been recently discussed controversially to play a causative role in the development of Parkinson's disease [Mittal et al., Science 357, 891-898 (2017) thereby representing a questionable therapeutic approach. Deep brain stimulation (DBS) is a powerful but invasive treatment option, and MRI guided hi-frequency ultrasound thalamotomy (MRgHIFU) is effective but induces permanent lesions in the brain. Both are only available at highly specialized neurosurgery centres to patients whose tremor is severely incapacitating.

Treatment options for tremor in general focus on conservative measures (drug therapy), however the unconscious mechanisms of tremor can hardly be treated adequately in patients with progressive neurological diseases as in Parkinson's disease. Therefore, treatment of tremor was focused on the reduction of the amplitude of the oscillation. Earliest approaches used ethanol (well known recreational substance acting inhibitory on glutamatergic inhibitory nerves) as this primarily recreational substance was used as a doping in sports necessitating a calm hand (e.g. in Olympic disciplines of shooting). The antihypertensive and antiarrhythmic drug Propranolol (beta adrenergic blocker) was later used as anti-tremor drug among others. Several derivatives of beta-adrenergic blockers from the drug group of anti-hypertensives were tested and used off label in this indication.

Neurosurgical treatment options provide disruptive solutions for brain centres of the thalamus being known to be involved in tremor circuits, destruction by thalamotomy, Magnet resonance guided high frequency ultrasound therapy are current solutions. Deep Brain Stimulation of specific brain areas is also reported to be effective to treat tremulous conditions in a limited number of patients.

Another treatment alternative is Botulinum toxin A, which is used in a broad range of different diseases and medical conditions, including cervical dystonia, spasticity, incontinency, migraine etc. Botulinum toxin A was also used in the treatment of e.g. dystonic tremor, task specific tremors, Parkinson tremor and Essential tremor. An intramuscular injection of Botulinum Toxin A reduces muscle tonus in treated muscles, and thereby reduces tremor amplitude by causing localized partial weakness of the involved muscles.

Local therapy of tremor by applying injections of a botulinum neurotoxin into tremulous muscles is always challenging as the tremor can affect the whole arm or just parts of it causing a wide range of clinical phenomena, depending on the underlying disease and tremor pattern. Tremor of the upper limb treated by administering the botulinum neurotoxin in a fixed dose/fixed muscle approach to several muscles of the wrist and forearm (primarily into wrist flexors and extensors) showed strong efficacy but also debilitating side effects. Adverse events were mainly weakness of the arm, wrist and dropping fingers. Those could be partially reduced by dose reduction in follow-up treatments, by modifying the selection of injected muscles or by switching the strategy to low initial starting doses followed by slow up-titration or booster injections. On the other hand, these changes may result in a reduced efficacy. Any low initial starting dose requires an early follow-up treatment (booster injections) to counteract insufficient initial treatment of the tremor. Modification of the selection of injected muscles intended to modify the weakness pattern of the limb, could cause further weakness of other muscles, thereby making the treatment for patients less tolerable.

The muscle selection, depending on the range of motions during tremor, is also difficult, as ideally this should be decided based on precise analysis of the muscle function and the sum thereof. Visual clinical assessment of the movement direction and amplitude of the oscillation is unprecise and limited by poor discriminative ability of the examiner with respect to obscure components of the complex movement. However, without having more precise and reliable alternatives available, visual assessment is still used regularly to allocate muscles and doses, thereby focusing the attention to obvious movements of the wrist (most frequently flexion and extension). Therefore, early approaches used fixed dosages to a preselected set of muscles of the forearm (primarily flexors and extensors of the wrist). This might have led to an unexpectedly high frequency of muscular weakness of the wrist in early studies. However, the tremulous movement of the arm can be the result of a tremor of further muscles of the arm (elbow and shoulder muscles). The range of motion of these joints adds to the tremor of the wrist, which causes as a result a subsuming overall effect. Furthermore, other parts of the body can have also tremor, e.g. the lower limb, the head, the trunk, which might cause the instability of the body with oscillating waves transmitted towards distant body parts like the wrist. In upper limb tremor of the hands the tremulous movement can affect the proximal and distal muscle groups (wrist moving muscles and shoulder moving muscles) in parallel providing no typical oscillation pattern. Individual components (tremor of individual muscles, or even muscle groups) of the visually detected subsuming tremor can than hardly be differentiated by solely visual observation by the clinician.

There are technical measures available for differentiating muscle group contribution to tremor, like kinematic measurements or electromyography EMG. However, those techniques are not wide spread in clinical practice for analysing tremor. In particular, needle EMG is disadvantageous for a large number of muscles to be examined as this is a painful procedure. A further challenge is that training of injectors is not established and there are no generally accepted guidelines for muscle and dose selection strategies for treating tremor with botulinum toxin A. Selecting suitable muscles and the correct dose per muscle is crucial for treatment efficacy and safety of tremor treatments of the upper limb, lower limb, head and neck and vocal cords. There is currently no defined therapeutic dose window in total or per muscle which allows the optimal treatment with appropriate efficacy and tolerability. In particular, a possible weakness of botulinum neurotoxin treated muscles because of the general mode of action of botulinum neurotoxins, i.e. the muscle paralysis, needs to be taken into account.

In WO2015039244 a technique of treating tremor is presented which uses an adaptive and customary system for determining muscle activity by kinematic analysis of the upper limb (tremor amplitude mainly) and calculating the individual dosage being administered to individual muscles. Jog et al. demonstrated the applicability of this flexible dosage determination in a clinical trial using the botulinum neurotoxin A Xeomin® in 19 patients Jog et al. Poster Presented at TOXINS 2017, Madrid, Spain, 18-21 Jan. 2017).

There is a strong demand to further improve the therapeutic options available for treating tremor. In particular, it is desirable to treat effectively and without adverse events the upper-limb tremor in patients with Essential Tremor, Parkinson's disease and any other tremors of the upper limb.

### OBJECTS OF THE INVENTION

It was an object of the present invention to improve the treatment of tremor, in particular to provide a treatment of any tremor symptom of the upper limb. It was furthermore an object of the invention to provide a safe and efficacious treatment scheme for treating a tremor of the upper limb which is easy to administer to the patient without detailed tremor analysis and comprehensive tremor decomposition. It was a particular object of the present invention to develop a dosing system to administer a botulinum neurotoxin for use in treating a tremor of the upper limbs, which allows an adaptive and customized administration to the muscles of the wrist and forearm in combination with an easy-to-administer fixed administration to the muscles of the elbow and the shoulder.

### SUMMARY OF THE INVENTION

Surprisingly, it has been identified that a botulinum neurotoxin may advantageously be used to provide a treatment of a tremor of the upper limb, if the botulinum neurotoxin is administered to at least one muscle of the forearm/wrist, elbow and shoulder, wherein the botulinum neurotoxin is administered to at least one muscle of the forearm/wrist selected from the group of M. extensor carpi ulnaris (ECU), M. extensor carpi radialis ECR), M. pronator quadratus (PQ) and M. supinator in a dosage in the range of 2 to 6 U and wherein the botulinum neurotoxin is administered to at least one muscle of the elbow in a dosage of about 20 U and to at least one muscle of the shoulder in a dosage of about 15 U.

Thus, in one aspect, the present invention relates to a botulinum neurotoxin for use in treating a tremor of the upper limb comprising the administration of a botulinum neurotoxin to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of 2 to 6 U and wherein the botulinum neurotoxin is administered to at least one muscle of the elbow in a dosage of about 20 U and to at least one muscle of the shoulder in a dosage of about 15 U.

In another aspect, the present invention relates to a pharmaceutical composition comprising a botulinum neurotoxin for use in treating a tremor of the upper limb comprising the administration of a botulinum neurotoxin to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of 2 to 6 U and wherein the botulinum neurotoxin is administered to at least one muscle of the elbow in a dosage of about 20 U and to at least one muscle of the shoulder in a dosage of about 15 U.

In yet another aspect, the present invention relates to a method of treating a disease or condition associated with a tremor of the upper limb comprising the administration of a botulinum neurotoxin to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of 2 to 6 U and wherein the botulinum neurotoxin is administered to at least one muscle of the elbow in a dosage of about 20 U and to at least one muscle of the shoulder in a dosage of about 15 U.

### DETAILED DESCRIPTION OF THE INVENTION

The present invention may be understood more readily by reference to the following detailed description of the invention and the example included therein.

In one aspect, the invention relates to a botulinum neurotoxin for use in treating a tremor of the upper limb comprising the administration of a botulinum neurotoxin to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of about 2 to 6 U and wherein the botulinum neurotoxin is administered to at least one muscle of the elbow in a dosage of about 20 U and to at least one muscle of the shoulder in a dosage of about 15 U.

In a preferred embodiment of the present invention, the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of about 2.5 to 5 U and wherein the botulinum neurotoxin is administered to at least one muscle of the elbow in a dosage of about 20 U and to at least one muscle of the shoulder in a dosage of about 15 U.

In a further preferred embodiment of the present invention, the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage of about 2.5 U and wherein the botulinum neurotoxin is administered to at least one muscle of the elbow in a dosage of about 20 U and to at least one muscle of the shoulder in a dosage of about 15 U.

In an embodiment of the present invention, the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of about 2 to 6 U and to at least one muscle of the wrist/forearm selected from the group of M.flexor carpi radialis (FCR), M.flexor carpi ulnaris (FCU) and M.pronator teres (PT) in a dose in the range of 4 to 16 U per muscle, and wherein the botulinum neurotoxin is administered to at least one muscle of the elbow in a dosage of about 20 U and to at least one muscle of the shoulder in a dosage of about 15 U.

In a further embodiment of the present invention, the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage of about 2.5 U and to at least one muscles of the wrist/forearm selected from the group of M.flexor carpi radialis (FCR), M.flexor carpi ulnaris (FCU) and M.pronator teres (PT) in a dose of about 10 U per muscle, and wherein the botulinum neurotoxin is administered to at least one muscle of the elbow in a dosage of about 20 U and to at least one muscle of the shoulder in a dosage of about 15 U.

In one aspect of the present invention, the botulinum neurotoxin for use in treating a tremor of the upper limb is administered to at least one muscle of the forearm/wrist, elbow and shoulder, wherein the botulinum neurotoxin is administered to 4, 5, 6 or 7 muscles of the forearm/wrist selected from the group of M.extensor carpi ulnaris (ECU), M.extensor carpi radialis (ECR), M.pronator quadratus (PQ), M.supinator, M.flexor carpi radialis (FCR), M.flexor carpi ulnaris (FCU) and M.pronator teres (PT), and wherein the dosage administered the M.extensor carpi ulnaris (ECU), M.extensor carpi radialis (ECR) and M.pronator quadratus (PQ) and M.supinator is in the range of about 2 to 6 U and the dosage administered to the M.flexor carpi radialis (FCR), M.flexor carpi ulnaris (FCU) and M.pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U.

In a preferred embodiment of the present invention, the botulinum neurotoxin for use in treating a tremor of the upper limb is administered to at least one muscle of the forearm/wrist, elbow and shoulder, wherein the botulinum neurotoxin is administered to 5 muscles of the forearm/wrist selected from the group of M. extensor carpi radialis (ECR), M. supinator, M. flexor carpi radialis (FCR), M. flexor carpi ulnaris (FCU) and M. pronator teres (PT), and wherein the dosage administered M. extensor carpi radialis (ECR)) and M. supinator is in the range of about 2 to 6 U and the dosage administered to the M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U.

In a further aspect of the present invention, the botulinum neurotoxin for use in treating a tremor of the upper limb is administered to at least one muscle of the forearm/wrist, elbow and shoulder, wherein the botulinum neurotoxin is administered to 4, 5, 6 or 7 muscles of the forearm/wrist selected from the group of M. extensor carpi ulnaris (ECU), M. extensor carpi radialis (ECR), M. pronator quadratus (PQ), M. supinator, M. flexor carpi radialis (FCR), M. flexor carpi ulnaris (FCU) and M. pronator teres (PT), and wherein the dosage administered the M. extensor carpi ulnaris (ECU), M. extensor carpi radialis (ECR) and M. pronator quadratus (PQ) and M. supinator is in the range of about 2 to 6 U and the dosage administered to the M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U, and wherein the botulinum neurotoxin is not administered to the M. externsor digitorium communis (EDC), the M. biceps brachii, the deltoid muscle and the M. teres major.

In a further preferred embodiment of the present invention, the botulinum neurotoxin for use in treating a tremor of the upper limb is administered to at least one muscle of the forearm/wrist, elbow and shoulder, wherein the botulinum neurotoxin is administered to 5 muscles of the forearm/wrist selected from the group of M. extensor carpi radialis (ECR), M. supinator, M. flexor carpi radialis (FCR), M. flexor carpi ulnaris (FCU) and M. pronator teres (PT), and wherein the dosage administered to M. extensor carpi radialis (ECR) and M. supinator is in the range of about 2 to 6 U and the dosage administered to M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U and wherein the botulinum neurotoxin is not administered to M. externsor digitorium communis (EDC), the M. biceps brachii, the deltoid muscle and the M. teres major.

In a preferred embodiment of the present invention, the botulinum neurotoxin for use in treating a tremor of the upper limb is administered to at least one muscle of the forearm/wrist, elbow and shoulder, wherein the botulinum neurotoxin is administered to 4, 5, 6 or 7 muscles of the forearm/wrist selected from the group of M.extensor carpi ulnaris (ECU), M.extensor carpi radialis (ECR), M.pronator quadratus (PQ), M.supinator, M.flexor carpi radialis (FCR), M.flexor carpi ulnaris (FCU) and M.pronator teres (PT), and wherein the dosage administered the M.extensor carpi ulnaris (ECU), M.extensor carpi radialis (ECR), M.pronator quadratus (PQ) and M.supinator is in the range of about 2.5 to 5 U and the dosage administered to the M.flexor carpi radialis (FCR), M.flexor carpi ulnaris (FCU) and M.pronator teres (PT) is in the range of about 5 to 15 U per muscle, and wherein the botulinum neurotoxin is administered to at least one muscle of the elbow in a dosage of about 20 U and to at least one muscle of the shoulder in a dosage of about 15 U.

In a preferred embodiment of the present invention, the botulinum neurotoxin for use in treating a tremor of the upper limb is administered to at least one muscle of the forearm/wrist, elbow and shoulder, wherein the botulinum neurotoxin is administered to 5 muscles of the forearm/wrist selected from the group of M. extensor carpi radialis (ECR), M. supinator, M. flexor carpi radialis (FCR), M. flexor carpi ulnaris (FCU) and M. pronator teres (PT), and wherein the dosage administered to M. extensor carpi radialis (ECR)) and M. supinator is in the range of about 2 to 5 U and the dosage administered to M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U.

In a preferred embodiment of the present invention, the botulinum neurotoxin for use in treating a tremor of the upper limb is administered to at least one muscle of the forearm/wrist, elbow and shoulder, wherein the botulinum neurotoxin is administered to 4, 5, 6 or 7 muscles of the forearm/wrist selected from the group of M. extensor carpi ulnaris (ECU), M. extensor carpi radialis (ECR), M. pronator quadratus (PQ), M. supinator, M. flexor carpi radialis (FCR), M. flexor carpi ulnaris (FCU) and M. pronator teres (PT), and wherein the dosage administered the M. extensor carpi ulnaris (ECU), M. extensor carpi radialis (ECR), M. pronator quadratus (PQ) and M. supinator is in the range of about 2.5 to 5 U and the dosage administered to the M. flexor carpi radialis (FCR), M. flexor carpi ulnaris (FCU) and M. pronator teres (PT) is in the range of about 5 to 15 U per muscle, and wherein the botulinum neurotoxin is administered to at least one muscle of the elbow in a dosage of about 20 U and to at least one muscle of the shoulder in a dosage of about 15 U, and wherein the botulinum neurotoxin is not administered to the M. externsor digitorium communis (EDC), the M. biceps brachii, the deltoid muscle and the M. teres major.

In a further preferred embodiment of the present invention, the botulinum neurotoxin for use in treating a tremor of the upper limb is administered to at least one muscle of the forearm/wrist, elbow and shoulder, wherein the botulinum neurotoxin is administered to 5 muscles of the forearm/wrist selected from the group of M. extensor carpi radialis (ECR), M. supinator, M. flexor carpi radialis (FCR), M. flexor carpi ulnaris (FCU) and M. pronator teres (PT), and wherein the dosage administered to M. extensor carpi radialis (ECR)) and M. supinator is in the range of about 2 to 5 U and the dosage administered to the M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U and wherein the botulinum neurotoxin is not administered to the M. externsor digitorium communis (EDC), the M. biceps brachii, the deltoid muscle and the M. teres major.

In a particular preferred embodiment of the present invention, the botulinum neurotoxin for use in treating a tremor of the upper limb is administered to at least one muscle of the forearm/wrist, elbow and shoulder, wherein the botulinum neurotoxin is administered to 4, 5, 6 or 7 muscles of the forearm/wrist selected from the group of M. extensor carpi ulnaris (ECU), M. extensor carpi radialis (ECR), M. pronator quadratus (PQ), M. supinator, M. flexor carpi radialis (FCR), M. flexor carpi ulnaris (FCU) and M. pronator teres (PT), and wherein the dosage administered the M. extensor carpi ulnaris (ECU), M. extensor carpi radialis (ECR), M. pronator quadratus (PQ) and M. supinator is in the range of about 2.5 to 5 U and the dosage administered to the M. flexor carpi radialis (FCR), M. flexor carpi ulnaris (FCU) and M. pronator teres (PT) is in the range of about 5 to 15 U per muscle, and wherein the botulinum neurotoxin is administered to at least one muscle of the elbow in a dosage of about 20 U and to at least one muscle of the shoulder in a dosage of about 15 U, and wherein the total dosage of botulinum neurotoxin administered to the muscles of the forearm/wrist does not exceed 65 U.

In a further preferred embodiment of the present invention, the botulinum neurotoxin for use in treating a tremor of the upper limb is administered to at least one muscle of the forearm/wrist, elbow and shoulder, wherein the botulinum neurotoxin is administered to 5 muscles of the forearm/wrist selected from the group of M. extensor carpi radialis (ECR), M. supinator, M. flexor carpi radialis (FCR), M. flexor carpi ulnaris (FCU) and M. pronator teres (PT), and wherein the dosage administered to M. extensor carpi radialis (ECR) and M. supinator is in the range of about 2 to 5 U and the dosage administered to M. flexor carpi radialis (FCR), M. flexor carpi ulnaris (FCU) and M. pronator teres (PT) is in the range of about 4 to 16 U per muscle, and wherein the botulinum neurotoxin is administered to at least one muscle of the elbow in a dosage of about 20 U and to at least one muscle of the shoulder in a dosage of about 15 U, and wherein the total dosage of botulinum neurotoxin administered to the muscles of the forearm/wrist does not exceed 65 U.

In another aspect of the present invention, the botulinum neurotoxin for use in treating a tremor of the upper limb is administered to at least one muscle of the forearm/wrist, elbow and shoulder, wherein the botulinum neurotoxin is administered to 7 muscles of the forearm/wrist selected from the group of M.extensor carpi ulnaris (ECU), M.extensor carpi radialis (ECR), M.pronator quadratus (PQ), M.supinator, M.flexor carpi radialis (FCR), M.flexor carpi ulnaris (FCU) and M.pronator teres (PT), and wherein the dosage administered the M.extensor carpi ulnaris (ECU), M.extensor carpi radialis (ECR), M.pronator quadratus (PQ) and M.supinator is about 2.5 U and the dosage administered to the M.flexor carpi radialis (FCR), M.flexor carpi ulnaris (FCU) and M.pronator teres (PT) is about 10 U per muscle, and wherein the botulinum neurotoxin is administered to at least one muscle of the elbow in a dosage of about 20 U and to at least one muscle of the shoulder in a dosage of about 15 U.

In a further aspect of the present invention, the botulinum neurotoxin for use in treating a tremor of the upper limb is administered to at least one muscle of the forearm/wrist, elbow and shoulder, wherein the botulinum neurotoxin is administered to 7 muscles of the forearm/wrist selected from the group of M. extensor carpi ulnaris (ECU), M. extensor carpi radialis (ECR), M. pronator quadratus (PQ), M. supinator, M. flexor carpi radialis (FCR), M. flexor carpi ulnaris (FCU) and M. pronator teres (PT), and wherein the dosage administered the M. extensor carpi ulnaris (ECU), M. extensor carpi radialis (ECR), M. pronator quadratus (PQ) and M. supinator is about 2.5 U and the dosage administered to the M. flexor carpi radialis (FCR), M. flexor carpi ulnaris (FCU) and M. pronator teres (PT) is about 10 U per muscle, and wherein the botulinum neurotoxin is administered to at least one muscle of the elbow in a dosage of about 20 U and to at least one muscle of the shoulder in a dosage of about 15 U, and wherein the botulinum neurotoxin is not administered to the M. externsor digitorium communis (EDC), the M. biceps brachii, the deltoid muscle and the M. teres major.

In a particular preferred embodiment of the present invention, the botulinum neurotoxin for use in treating a tremor of the upper limb is administered to at least one muscle of the forearm/wrist, elbow and shoulder, wherein the botulinum neurotoxin is administered to 7 muscles of the forearm/wrist selected from the group of M.extensor carpi ulnaris (ECU), M.extensor carpi radialis (ECR), M.pronator quadratus (PQ), M.supinator, M.flexor carpi radialis (FCR), M.flexor carpi ulnaris (FCU) and M.pronator teres (PT), and wherein the dosage administered the M.extensor carpi ulnaris (ECU), M.extensor carpi radialis (ECR), M.pronator quadratus (PQ) and M.supinator is about 2.5 U and the dosage administered to the M.flexor carpi radialis (FCR), M.flexor carpi ulnaris (FCU) and M.pronator teres (PT) is about 10 U per muscle, and wherein the botulinum neurotoxin is administered to at least one muscle of the elbow in a dosage of about 20 U and to at least one muscle of the shoulder in a dosage of about 15 U, and wherein the total dosage of the botulinum neurotoxin administered to the muscles of the forearm/wrist does not exceed 40 U.

In the context of the present invention the tremor of the upper limb can be tremor in patients with essential tremor, tremor due to neurodegenerative diseases such as Parkinson's disease, dystonic tremor, cerebellar tremor, and any other tremors of the upper limb (e.g. musician's tremor, Holmes tremor, neuropathic tremor, task- or position specific tremor, rest tremor, action tremor, etc.).

Further, tremor symptoms and syndromes in childhood and adolescence are in the context of the present invention. In those patients the dosing of the treatment should be oriented to the body weight of the child treated. Adapted doses are in the range of 1-8 U/kg BW, (maximum of 140 U) per upper limb.

Generally, it is envisaged according to the present invention that the botulinum neurotoxin for use is administered to the forearm/wrist muscles in a total dose in the range of between 30 U to 65 U. In a more specific embodiment it is envisaged that the botulinum neurotoxin for use is administered to the forearm/wrist muscles in a total dose in the range of between 40 U to 65 U. In particular embodiments of the present invention, the botulinum neurotoxin for use is administered to the forearm/wrist muscles in a total dose that does not exceed 65 U. In a further embodiment of the present invention, the botulinum neurotoxin for use is administered to the forearm/wrist muscles in a total dose that does not exceed 40 U. In a preferred embodiment of the present invention, the botulinum neurotoxin for use is administered to wrist/forearm extensor and flexor muscles in a dose ratio in the range of 1:2 to 1:6.

Generally, it is envisaged according to the present invention, that the botulinum neurotoxin for use is administered to at least one muscle of each the forearm/wrist, the elbow and the shoulder.

In a further aspect of the present invention, M. extensor carpi radialis (ECR) can be divided in M. extensor carpi radialis longus and M. extensor carpi radialis brevis.

In a further embodiment of the present invention the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M. extensor carpi ulnaris (ECU), M. pronator quadratus (PQ), M. extensor carpi radialis longus, M. extensor carpi radialis brevis and M. supinator and wherein the botulinum neurotoxin is administered to the M. extensor carpi ulnaris (ECU), M. pronator quadratus (PQ) and M. supinator in a dosage in the range of about 2.5 to 6 U, to M. extensor carpi radialis longus in a dose in the range of between 1.25 U and 3 U and to the M. extensor carpi radialis brevis in a dose in the range of between 1.25 U and 3 U and to at least one muscle of the wrist/forearm selected from the group of M. flexor carpi radialis (FCR), M. flexor carpi ulnaris (FCU) and M. pronator teres (PT) in a dose in the range of 4 to 16 U per muscle and wherein the botulinum neurotoxin is administered to at least one muscle of the elbow in a dosage of about 20 U and to at least one muscle of the shoulder in a dosage of about 15 U.

In a further embodiment of the present invention, the botulinum neurotoxin for use in treating a tremor of the upper limb is administered to at least one muscle of the forearm/wrist, elbow and shoulder, wherein the botulinum neurotoxin is administered to at least one muscle of the forearm/wrist selected from the group of M. extensor carpi ulnaris (ECU), M. pronator quadratus (PQ), M. extensor carpi radialis longus, M. extensor carpi radialis brevis and M. supinator and wherein the botulinum neurotoxin is administered to the M. extensor carpi ulnaris (ECU), M. pronator quadratus (PQ) and M. supinator in a dosage in the range of about 2.5 to 5 U, to M. extensor carpi radialis longus in a dose of 1.25 U and to the M. extensor carpi radialis brevis in a dose of 1.25 U and to at least one muscle of the wrist/forearm selected from the group of M. flexor carpi radialis (FCR), M. flexor carpi ulnaris (FCU) and M. pronator teres (PT) in a dose in the range of 4 to 16 U per muscle and wherein the botulinum neurotoxin is administered to at least one muscle of the elbow in a dosage of about 20 U and to at least one muscle of the shoulder in a dosage of about 15 U.

According to the present invention, the botulinum neurotoxin for use is not administered to the muscles of the forearm/wrist selected from the group of M. flexor digitorum superficialis and profundus, M. palmaris longus, M. flexor pollicis longus, M. extensor pollicis brevis and longus, M. abductor pollicis, M. opponens pollicis, M. abductor pollicis brevis, M. flexor pollicis brevis. M. adductor pollicis. M. interossei dorsales, M. lumbricales, M. opponens digiti minimi, M. abductor digiti minimi and M. flexor digiti minimi brevis. In a preferred embodiment of the present invention, the botulinum neurotoxin for use is not administered to the M. externsor digitorium communis.

In further embodiments of the present invention, the botulinum neurotoxin for use is administered to at least one muscle of the elbow selected from the group of M. brachialis and M. triceps brachii. In a preferred embodiment, the botulinum neurotoxin for use is administered to at least one muscle of the elbow selected from the group of M. brachialis and M. triceps brachii in a dosage of about 20 U per muscle. In a particular preferred embodiment of the present invention, the botulinum neurotoxin for use is administered to the muscles of the elbow in a total dose that does not exceed 40 U.

In the context of the present invention, the botulinum neurotoxin for use is not administered to muscles of the elbow selected from M. brachioradialis, and M. anconeus. In a further embodiment of the present invention, the botulinum neurotoxin for use is not administered to the M. biceps brachii.

In further embodiments of the present invention, the botulinum toxin for use is administered to at least one muscle of the shoulder selected from the group of M. latissimus dorsi, M. pectoralis major, M. supraspinatus and M. infraspinatus. In a particular embodiment of the present invention, the botulinum toxin for use is administered to at least one muscle of the shoulder selected from the group of M. latissimus dorsi, M. pectoralis major, M. supraspinatus and M. infraspinatus in a dosage of about 15 U per muscle. In further embodiments of the present invention, the botulinum neurotoxin for use is administered to muscles of the shoulder wherein the total dose does not exceed 60 U.

In the context of the present invention the botulinum neurotoxin for use is not administered to a muscle of the shoulder selected from the group of M. coracobrachialis, M. pectoralis minor, M. subclavius, M. subscapularis, M. serratus anterior, M. levator scapulae, M. rhomboid minor and major and M. trapezius. In a further preferred embodiment of the present invention, the botulinum neurotoxin for use is not administered to the M. deltoideus and to the M. teres major muscle.

Generally, it is envisaged according to the present invention that the botulinum neurotoxin for use is administered to the muscles of the forearm/wrist, elbow and shoulder in a total dose in the range of between 130 U to 165 U. In a more specific embodiment it is envisaged that the botulinum neurotoxin for use is administered to the muscles of the forearm/wrist, elbow and shoulder in a total dose in the range of between 140 U to 165 U. In a preferred embodiment of the present invention, the botulinum neurotoxin for use is administered to the muscles of the forearm/wrist, elbow and shoulder in a total dose that does not exceed 165 U. In a further preferred embodiment of the present invention, the botulinum neurotoxin for use is administered to the muscles of the forearm/wrist, elbow and shoulder in a total dose that does not exceed 140 U. If the botulinum neurotoxin for use is administered bilaterally, the respective dosages are adjusted accordingly and are doubled compared to the unilateral treatment. Generally, it is envisaged according to the present invention that the botulinum neurotoxin for use is administered bilaterally to the muscles of the forearm/wrist, elbow and shoulder in a total dose in the range of between 260 U to 330 U. In a more specific embodiment it is envisaged that the botulinum neurotoxin for use is administered bilaterally to the muscles of the forearm/wrist, elbow and shoulder in a total dose in the range of between 280 U to 330 U. In a preferred embodiment of the present invention, the botulinum neurotoxin for use is administered bilaterally to the muscles of the forearm/wrist, elbow and shoulder in a total dose that does not exceed 330 U. In a further preferred embodiment of the present invention, the botulinum neurotoxin for use is administered bilaterally to the muscles of the forearm/wrist, elbow and shoulder in a total dose that does not exceed 280 U.

In a preferred embodiment, the botulinum neurotoxin for use is administered to at least one muscle of the forearm/wrist, at least one muscle of the elbow and least one muscle of the shoulder according to the dosing scheme:

| | |
|---|---|
| M. extensor carpi ulnaris (ECU) | about 2.5-5.0 U |
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. pronator quadratus (PQ) | about 2.5-5.0 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

In a particular preferred embodiment, the botulinum neurotoxin for use is administered to the 4, 5, 6, or 7 muscles of the forearm/wrist, 2 muscles of the elbow and 4 muscles of the shoulder according to the dosing scheme:

| | |
|---|---|
| M. extensor carpi ulnaris (ECU) | about 2.5-5.0 U |
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. pronator quadratus (PQ) | about 2.5-5.0 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

In a particular preferred embodiment, the botulinum neurotoxin for use is administered to 5 muscles of the forearm/wrist, 2 muscles of the elbow and 4 muscles of the shoulder according to the dosing scheme:

| | |
|---|---|
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

In a further preferred embodiment, the botulinum neurotoxin for use is administered to at least one muscle of the forearm/wrist, at least one muscle of the elbow and at least one muscle of the shoulder according to the dosing scheme:

| | |
|---|---|
| M. extensor carpi ulnaris (ECU) | about 2.5 U |
| M. extensor carpi radialis (ECR) | about 2.5 U |
| M. flexor carpi radialis (FCR) | about 10 U |
| M. flexor carpi ulnaris (FCU) | about 10 U |
| M. pronator teres (PT) | about 10 U |
| M. pronator quadratus (PQ) | about 2.5 U |
| M. supinator | about 2.5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

In a particular preferred embodiment, the botulinum neurotoxin for use is administered to 7 muscles of the forearm/wrist, 2 muscles of the elbow and 4 muscles of the shoulder according to the dosing scheme:

| | |
|---|---|
| M. extensor carpi ulnaris (ECU) | about 2.5 U |
| M. extensor carpi radialis (ECR) | about 2.5 U |
| M. flexor carpi radialis (FCR) | about 10 U |
| M. flexor carpi ulnaris (FCU) | about 10 U |
| M. pronator teres (PT) | about 10 U |
| M. pronator quadratus (PQ) | about 2.5 U |
| M. supinator | about 2.5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

In an alternative embodiment of the present invention, the botulinum neurotoxin for use is administered to at least one muscle of the forearm/wrist, at least one muscle of the elbow and least one muscle of the shoulder according to the dosing scheme:

| | |
|---|---|
| M. extensor carpi ulnaris (ECU) | about 2.5-5.0 U |
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. pronator quadratus (PQ) | about 2.5-5.0 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |
| M. deltoideus | about 10-15 U |

In a further alternative embodiment of the present invention, the botulinum neurotoxin for use is administered to the 4, 5, 6, or 7 muscles of the forearm/wrist, 2 muscles of the elbow and 4 muscles of the shoulder according to the dosing scheme:

| | |
|---|---|
| M. extensor carpi ulnaris (ECU) | about 2.5-5.0 U |
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. pronator quadratus (PQ) | about 2.5-5.0 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |
| M. deltoideus | about 10-15 U |

In a further alternative embodiment of the present invention, the botulinum neurotoxin for use is administered to 5 muscles of the forearm/wrist, 2 muscles of the elbow and 4 muscles of the shoulder according to the dosing scheme:

| | |
|---|---|
| M. extensor carpi radialis (ECR) | about 2.5-5.0 U |
| M. flexor carpi radialis (FCR) | about 5-15 U |
| M. flexor carpi ulnaris (FCU) | about 5-15 U |
| M. pronator teres (PT) | about 5-15 U |
| M. supinator | about 2.5-5.0 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |
| M. deltoideus | about 10-15 U |

In a further alternative embodiment of the present invention, the botulinum neurotoxin for use is administered to at least one muscle of the forearm/wrist, at least one muscle of the elbow and at least one muscle of the shoulder according to the dosing scheme:

| | |
|---|---|
| M. extensor carpi ulnaris (ECU) | about 2.5 U |
| M. extensor carpi radialis (ECR) | about 2.5 U |
| M. flexor carpi radialis (FCR) | about 10 U |
| M. flexor carpi ulnaris (FCU) | about 10 U |
| M. pronator teres (PT) | about 10 U |
| M. pronator quadratus (PQ) | about 2.5 U |
| M. supinator | about 2.5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |
| M. deltoideus | about 10-15 U |

In a further alternative embodiment of the present invention, the botulinum neurotoxin for use is administered to 7 muscles of the forearm/wrist, 2 muscles of the elbow and 5 muscles of the shoulder according to the dosing scheme:

| | |
|---|---|
| M. extensor carpi ulnaris (ECU) | about 2.5 U |
| M. extensor carpi radialis (ECR) | about 2.5 U |
| M. flexor carpi radialis (FCR) | about 10 U |
| M. flexor carpi ulnaris (FCU) | about 10 U |
| M. pronator teres (PT) | about 10 U |
| M. pronator quadratus (PQ) | about 2.5 U |
| M. supinator | about 2.5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |
| M. deltoideus | about 10-15 U |

In the context of the present invention, the botulinum neurotoxin for use can be administered unilateral or bilateral. In a preferred embodiment, the botulinum neurotoxin for use is administered bilaterally.

In particular embodiments of the present invention, the botulinum neurotoxin for use is administered to the muscles of the wrist/forearm, elbow and shoulder as aqueous reconstituted solution in a concentration in the range of 20 to 80 U/mL. In a preferred embodiment of the present invention, the botulinum neurotoxin for use is administered to the muscles of the wrist/forearm, elbow and shoulder as aqueous solution in a concentration in the range of 25 to 60 U/mL. In a particular preferred embodiment, the botulinum neurotoxin for use is administered to the muscles of the wrist/forearm, elbow and shoulder as aqueous solution in a concentration of 25 U/mL. It is generally understood, that the botulinum neurotoxin can be prepared as lyophilisate comprising the active agent together with pharmaceutically acceptable excipients. Prior use, the lyophilisate is reconstituted by adding a suitable amount of physiological saline or other suitable reconstitution media to provide the reconstituted botulinum neurotoxin solution in the intended concentration. Alternatively, the botulinum neurotoxin for use can be provided as a pre-filled syringe as it is disclosed, for example in WO2016/102068, WO 2016/124213 or WO2017/220553. The latter application form avoids any reconstitution step and allows manufacturing and providing the botulinum neurotoxin already suitably concentrated.

In the context of the present invention, the botulinum neurotoxin for use is administered to the wrist/forearm, elbow and shoulder muscles of the arm in a total volume of not more than 6.6 mL per arm. In a further embodiment of the present invention, the botulinum neurotoxin for use is administered to individual muscles in 0.1-0.8 mL per muscle.

Generally, the botulinum neurotoxin for use is administered to individual muscles in relation to muscle anatomy. The person skilled in the art is aware of methods, how the botulinum neurotoxin for use is administered into the anatomical location of motor units. Depending on the anatomical size, shape, physiologic strength and function of muscles the location and extent of neuro motor endplates to be affected by botulinum neurotoxin, the number of injection points per muscle vary. Anatomical atlases are used to define the location and extent of injections given to individual muscles. The injections of the botulinum neurotoxin for use can also be guided by using means of EMG, ultrasound or electrical stimulation of the respective muscles.

The number of injection sites is generally determined in accordance to the respective muscle size and the person skilled in the art is aware of how to administer a botulinum neurotoxin to a specific muscle. In the context of the present invention, the botulinum neurotoxin is injected into the muscles of the wrist/forearm selected from the group of M.extensor carpi ulnaris (ECU), M.extensor carpi radialis (ECR), M.flexor carpi radialis (FCR), M.flexor carpi ulnaris (FCU), M.pronator teres (PT), M.pronator quadratus (PQ) and M.supinator by using one injection site for each muscle. The botulinum neurotoxin is injected into the muscles of the elbow selected from M. brachialis and M. triceps brachii by using two sites for each muscle. The botulinum neurotoxin is injected into the muscles of the shoulder selected from M. latissimus dorsi and M. pectoralis major by using two sites of for each muscle. The botulinum neurotoxin is injected into the muscles of the shoulder selected from M. supraspinatus and M. infraspinatus, by using three sites for each muscle.

Tremor severity is assessed according to the present invention by the Essential Tremor Rating Assessment Scale (TETRAS). This assessment uses a validated clinical scale designed specifically for the assessment of ET severity. It is rated 0-4 in half-point intervals for the head, face including jaw, voice, upper limb, lower limb worse side, while standing. The scale focuses on assessment of upper limb action tremor using the following subcategories: handwriting on the dominant side only; separate assessments on both sides for the following conditions: posture using arms forward outstretched and wing beat position, kinetic using finger to nose test, drawing of Archimedes spirals, and dot approximation in which a pen is held as close as possible to a dot on a piece paper without touching it. The TETRAS system has anchors that span a larger range of tremor amplitudes (>20 cm=grade 4), making it suitable for assessing patients with severe ET. Sum score of all items of the TETRAS are assessed to characterize the tremor severity. The upper limb motor performance subscore is rated by an experienced physician based on the tasks performed by the subjects (instructed by the investigator). The performance subscale allows 0.5-point increments in scoring. The 0.5-point increments in upper limb tremor ratings are defined by specific ranges of tremor amplitude. The minimum detectable change of TETRAS Performance scale is 8.9% of the baseline measure [Voller et al. Mov Disord. 2015].

In the context of the present invention the botulinum neurotoxin for use improves the tremor severity according to the TETRAS upper limb motor performance subscore scale rated by the investigator by at least 9.0%. In a preferred embodiment the botulinum neurotoxin for use improves the tremor severity according to the TETRAS upper limb motor performance subscore scale rated by the investigator by at least 15%. In a more preferred embodiment the botulinum neurotoxin for use improves the tremor severity according to the TETRAS upper limb motor performance subscore scale rated by the investigator by at least 25%. In a most preferred embodiment the botulinum neurotoxin for use improves the tremor severity according to the TETRAS upper limb motor performance subscore scale rated by the investigator by at least 40%.

Alternatively tremor severity can be assessed according to the present invention by using standardized computerized kinematic tremor analysis. This analysis assesses tremor intensity by measuring angular tremor amplitude in relation to functional groups of muscles at the shoulder (flexion/extension, adduction/abduction), elbow (flexion/extension) and wrist/forearm level (flexion/extension, radial deviation/ulnar deviation, pronation/supination) for a series of 3 analysis trials for each of the following tasks: I. Posture 1: Shoulders flexed at 90° with forearms extended anteriorly and pronated; II. Posture 2: Shoulders flexed at 90° with forearms extended anteriorly in neutral position; Ill. Posture 3: Shoulders flexed at 90° with elbows also flexed and hands positioned near mouth; IV. Load 1: Holding an empty plastic cup (33 g); V. Load 2: Holding the same plastic cup filled with 1 pound in weight (355 ml soda can); VI. Load 3: Holding a cellphone (this would mimic holding a different object). Assessments of all three loads are in a posture with shoulders flexed at 90° and with forearms extended anteriorly.

In the context of the present invention, the botulinum neurotoxin for use decreases the maximum log-transformed accelerometric hand tremor amplitude from baseline versus placebo at week 4 by at least −0.10 m/s² (least squares [LS] mean difference [botulinum neurotoxin A vs. placebo]). In a preferred embodiment of the present invention, the botulinum neurotoxin for use decreases the maximum log-transformed accelerometric hand tremor amplitude from baseline versus placebo at week 4 by at least −0.10 m/s² (least squares [LS] mean difference [botulinum neurotoxin A vs. placebo]), and at week 6 by at least −0.10 m/s² (least squares [LS] mean difference [botulinum neurotoxin A vs. placebo]). In a more preferred embodiment of the present invention, the botulinum neurotoxin for use decreases the maximum log-transformed accelerometric hand tremor amplitude from baseline versus placebo at week 4 by at least −0.10 m/s² (least squares [LS] mean difference [botulinum neurotoxin A vs. placebo]), at week 6 by at least −0.10 m/s² (least squares [LS] mean difference [botulinum neurotoxin A vs. placebo]) and at week 8 by at least −0.10 m/s² (least squares [LS] mean difference [botulinum neurotoxin A vs. placebo]). In a preferred aspect of the present invention, the botulinum neurotoxin for use decreases the maximum log-transformed accelerometric hand tremor amplitude from baseline versus placebo at week 4 by at least −0.20 m/s² (least squares [LS] mean difference [botulinum neurotoxin A vs. placebo]). In a preferred embodiment of the present invention, the botulinum neurotoxin for use decreases the maximum log-transformed accelerometric hand tremor amplitude from baseline versus placebo at week 4 by at least −0.20 m/s² (least squares [LS] mean difference [botulinum neurotoxin A vs. placebo]), and at week 6 by at least −0.20 m/s² (least squares [LS] mean difference [botulinum neurotoxin A vs. placebo]). In a more preferred embodiment of the present invention, the botulinum neurotoxin for use decreases the maximum log-transformed accelerometric hand tremor amplitude from baseline versus placebo at Week 4 by at least −0.20 m/s² (least squares [LS] mean difference [botulinum neurotoxin A vs. placebo]), and at week 6 by at least −0.20 m/s² (least squares [LS] mean difference [botulinum neurotoxin A vs. placebo]) and at week 8 by at least −0.20 m/s² (least squares [LS] mean difference [botulinum neurotoxin A vs. placebo]). In a further aspect of the present invention the botulinum neurotoxin for use decreases the maximum wrist angular tremor amplitude from baseline versus placebo at week 4 by −0.7 degrees (LS mean difference [botulinum neurotoxin A vs. placebo]). In a preferred embodiment of the present invention the botulinum neurotoxin for use decreases the maximum wrist angular tremor amplitude from baseline versus placebo at week 4 by −0.7 degrees (LS mean difference [botulinum neurotoxin A vs. placebo]) and at week 6 by −0.7 degrees ((LS mean difference [botulinum neurotoxin A vs. placebo]). In a more preferred embodiment of the present invention the botulinum neurotoxin for use decreases the maximum wrist angular tremor amplitude from baseline versus placebo at week 4 by −0.7 degrees (LS mean difference [botulinum neurotoxin A vs. placebo]), at week 6 by −0.7 degrees ((LS mean difference [botulinum neurotoxin A vs. placebo]) and at week 8 by −0.7 degrees ((LS mean difference [botulinum neurotoxin A vs. placebo]). In a further preferred aspect of the present invention the botulinum neurotoxin for use decreases the maximum wrist angular tremor amplitude from baseline versus placebo at week 4 by −0.14 degrees (LS mean difference [botulinum neurotoxin A vs. placebo]). In a preferred embodiment of the present invention the botulinum neurotoxin for use decreases the maximum wrist angular tremor amplitude from baseline versus placebo at week 4 by −0.14 degrees (LS mean difference [botulinum neurotoxin A vs. placebo]) and at week 6 by −0.14 degrees ((LS mean difference [botulinum neurotoxin A vs. placebo]). In a more preferred embodiment of the present invention the botulinum neurotoxin for use decreases the maximum wrist angular tremor amplitude from baseline versus placebo at week 4 by −0.14 degrees (LS mean difference [botulinum neurotoxin A vs. placebo]), at week 6 by −0.14 degrees ((LS mean difference [botulinum neurotoxin A vs. placebo]) and at week 8 by −0.14 degrees ((LS mean difference [botulinum neurotoxin A vs. placebo]).

In vivo assays for assessing biological activity of a botulinum neurotoxin include the mouse LD50 assay and the ex vivo mouse hemidiaphragm assay as described by Pearce et al. (Pearce 1994, Toxicol. Appl. Pharmacol. 128: 69-77) and Dressler et al. (Dressler 2005, Mov. Disord. 20:1617-1619, Keller 2006, Neuroscience 139: 629-637) or a cell-based assay as described in WO2009/114748, WO2014/207109 or WO 2013/049508. The biological activity is commonly expressed in Mouse Units (U). As used herein, 1 U is the amount of neurotoxic component of the botulinum neurotoxin, which kills 50% of a specified mouse population after intraperitoneal injection, i.e. the mouse i.p. LD50. A particular useful method for determining the biological activity of a botulinum neurotoxin is a cell-based assay as it is disclosed for example in WO2009/114748, WO 2013/049508 or WO 2014/207109. The activity results obtained with such cell-based assays correspond to the activity values obtained in the mouse i.p. LD50 assay. Activity results obtained for Botulinum serotype A formulations like commercially available lncobotulinumtoxin A (Botulinumtoxin serotype A, without complexing proteins, Xeomin®, Merz Pharmaceuticals GmbH)) or Onabotulinumtoxin A (Botulinumtoxin serotype A, with complexing proteins, Botox®, Allergan Inc.) can be converted to values for other toxins using conversion rates known to the person skilled in the art. For example, the necessary dose of Abobotulinumtoxin A (Botulinumtoxin serotype A, with complexing proteins, Dysport®, Ipsen Biopharm Limited) can be determined by multiplication of the dose of lncobotulinumtoxin A or Onabotulinumtoxin A with a factor of 2.5 to 5. The dose for Rimabotulinumtoxin B (Botulinumtoxin serotype B, Myobloc®, Solstice Neurosciences/US WorldMeds LLC) can be calculated by multiplication of the dose of lncobotulinumtoxin A or Onabotulinumtoxin A with a factor of 20 to 40.

In the context of the present invention, the term “botulinum neurotoxin” refers to a natural neurotoxin obtainable from bacteria Clostridium botulinum or to a neurotoxin obtainable from alternative sources, including from recombinant technologies or from genetic or chemical modification. Particularly, the botulinum neurotoxins have endopeptidase activity. In the context of the present invention the term “botulinum toxin” and “botulinum neurotoxin” are used synonymously and are interchangeable.

In particular embodiments, the botulinum neurotoxin according to the invention is a botulinum neurotoxin complex.

In the context of the present invention, the terms “toxin complex” or “botulinum toxin complex” or “botulinum neurotoxin complex” are interchangeable and refer to a high molecular weight complex comprising the neurotoxic component of approximately 150 kDa and, in addition, non-toxic proteins of Clostridium botulinum, including hemagglutinin and non-hemagglutinin proteins (Sakaguchi 1983; Sugiyama 1980). Botulinum toxins, when released from lysed Clostridium cultures are generally associated with other bacterial proteins, which together form of a toxin complex. This complex usually contains additional, so-called “non-toxic” proteins, which will be referred here to as “complexing proteins” or “bacterial proteins”. The complex of neurotoxic component and bacterial proteins is referred to as “Clostridium botulinum toxin complex” or “botulinum toxin complex”. The molecular weight of this complex may vary from about 300,000 to about 900,000 Da. It is commercially available as Botulinum toxin A protein complex, for example, under the tradename BOTOX (Allergan Inc) or under the tradename DYSPORT (Ipsen Ltd).

In the context of the present invention, the terms “neurotoxic component” or “neurotoxin component” as used throughout the specification, relates to the subunit of the botulinum toxin complex which has a neurotoxic activity, and which has a molecular weight of approximately 150 kDa in serotype A. Unlike the toxin complex, the neurotoxic component in its isolated and pure form, i.e. devoid of any complexing Clostridium proteins, is acid labile and does not resist the aggressive environment in the gastrointestinal tract. A method for purifying and manufacturing the neurotoxic component of botulinum neurotoxin is demonstrated in U.S. Pat. No. 8,398,998. A high purity neurotoxic component, free of any complexing proteins, is for example available from Merz Pharmaceuticals GmbH, Frankfurt (Xeomin®).

The term “neurotoxic component” also includes the functional homologs found in the other serotypes of Clostridium botulinum.

In particular embodiments, the botulinum neurotoxin according to the invention is the neurotoxic component of a botulinum neurotoxin complex, wherein said neurotoxic component is devoid of any other protein component of the Clostridium botulinum neurotoxin complex.

In the context of the present invention, the term “devoid of any other protein component of the Clostridium botulinum neurotoxin complex” means without any non-toxic proteins of Clostridium botulinum, for example hemagglutinin proteins.

In particular embodiments, the botulinum neurotoxin according to the invention is selected from the group of different serotypes including botulinum neurotoxin serotype A (BoNT/A), botulinum neurotoxin serotype B (BoNT/B), botulinum neurotoxin serotype C1 (BoNT/C1), botulinum neurotoxin serotype D (BoNT/D), botulinum neurotoxin serotype E (BoNT/E), botulinum neurotoxin serotype F (BoNT/F) or botulinum neurotoxin serotype G (BoNT/G). The botulinum neurotoxin and, in particular, its light chain and heavy chain are derivable from one of the antigenically different serotypes of botulinum neurotoxins indicated above. In an aspect, said light and heavy chain of the botulinum neurotoxin are the light and heavy chain of a botulinum neurotoxin selected from the group consisting of: BoNT/A, BoNT/B, BoNT/C1, BoNT/D, BoNT/E, BoNT/F, or BoNT/G. In another aspect, a polynucleotide encoding said botulinum neurotoxin of the present invention comprises a nucleic acid sequence as shown in SEQ ID NO: 1 (BoNT/A), SEQ ID NO: 3 (BoNT/B), SEQ ID NO: 5 (BoNT/C1), SEQ ID NO: 7 (BoNT/D), SEQ ID NO: 9 (BoNT/E), SEQ ID NO: 11 (BoNT/F), or SEQ ID NO: 13 (BoNT/G). Moreover, encompassed is, in an aspect, a polynucleotide comprising a nucleic acid sequence encoding an amino acid sequence as shown in any one of SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), or SEQ ID NO: 14 (BoNT/G). Further encompassed is in an aspect the means and methods of the present invention to produce a botulinum neurotoxin comprising or consisting of an amino acid sequence selected from the group consisting of: SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), and SEQ ID NO: 14 (BoNT/G).

In another aspect, the said polynucleotide encoding a botulinum neurotoxin of the present invention is a variant of the aforementioned polynucleotides comprising one or more nucleotide substitutions, deletions and/or additions which in still another aspect may result in a polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 96%, at least 97%, at least 98% or at least 99% identical to the nucleic acid sequence as shown in any one of SEQ ID NOs: 1, 3, 5, 7, 9, 11, 13 or 15 or a nucleic acid sequence variant which encodes an amino acid sequence being at least 40%, at least 50%, at least 60%, at least 70%, at least 75%, at least 80%, at least 85%, at least 90%, at least 95%, at least 96%, at least 97%, at least 98% or at least 99% identical to the amino acid sequence as shown in any one of SEQ ID NOs: 2, 4, 6, 8, 10, 12, 14, or 16. The term “identical” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a polypeptide retaining one or more and, in another aspect, all of the biological properties of the respective botulinum neurotoxin, i.e. the BoNT/A, BoNT/B, BoNT/C1, BoNT/D, BoNT/E, BoNT/F or BoNT/G. Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In a further aspect, the variant polynucleotides can encode botulinum neurotoxins having improved or altered biological properties, e.g., they may comprise cleavage sites which are improved for enzyme recognition or may be improved for receptor binding or any other property specified above.

In a particular embodiment of the present invention, the botulinum neurotoxin for use in treating a tremor of the upper limb is administered together with at least one standard treatment selected from propranolol, primidone, any other antiepileptic or a calcium channel blocker or the botulinum neurotoxin is applied in parallel or sequentially to Deep Brain Stimulation or Magnetic Resonance guided High Frequency Ultrasound, local electrical stimulation, biofeedback, kinematic assessment guided stimulation, anti-tremor appliances, anti-tremor smartphone apps etc. treatment or combinations thereof.

In another aspect, the present invention relates to a pharmaceutical composition comprising a botulinum neurotoxin according to the invention for use in treating a tremor of the upper limb. For preparing a pharmaceutical preparation comprising a botulinum neurotoxin the neurotoxin can be formulated by various techniques dependent on the desired application purposes which are known in the art. For example, the (biologically active) botulinum neurotoxin can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid and the like. Exemplary of liquid carriers are glycerol, phosphate buffered saline solution, water, emulsions, various types of wetting agents, and the like. Suitable carriers comprise those mentioned above, and others well known in the art, see, e.g., Remington's Pharmaceutical Sciences, Mack Publishing Company, Easton, Pa. In an aspect, the pharmaceutical composition can be dissolved in a diluent, prior to administration. The diluent is also selected so as not to affect the biological activity of the botulinum neurotoxin product. Examples of such diluents are distilled water or physiological saline. In addition, the pharmaceutical composition or formulation may also include other carriers or non-toxic, non-therapeutic, non-immunogenic stabilizers and the like. Thus, the formulated botulinum neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid or free amino acids, such as methionine or histidine. In an aspect, suitable non-proteinaceous stabilizers are disclosed in WO 2005/007185, WO 2006/020208, WO2018/135722, WO2006/005910 or WO2012/134240. A suitable formulation for HSA-stabilized formulation comprising a botulinum neurotoxin according to the present invention is for example disclosed in U.S. Pat. No. 8,398,998 B2.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In the context of the present invention, the term “about” refers to the usual deviation of the dose of the botulinum neurotoxin actually administered to the muscle from the calculated dose. When the botulinum neurotoxin for use is administered as a reconstituted aqueous solution by using a syringe it is generally accepted by a person skilled in the art that this deviation is +1-10% of the calculated dose.

In particular embodiments, the pharmaceutical composition comprising a botulinum neurotoxin according to the invention is for use in treating a tremor of the upper limbs.

In another aspect, the present invention relates to a method of treating a tremor of the upper limbs wherein the method comprises the administration of a therapeutically effective amount of a botulinum neurotoxin according to the invention.

### EXAMPLE

### Example 1: General Treatment of Tremor of the Upper Limb Using NT201

NT 201 (active ingredient: NT 101, Botulinum neurotoxin type A, free from complexing proteins, US Adopted Name lncobotulinumtoxin A), excipients human serum albumin plus sucrose) is provided in vials for reconstitution. For the unilateral treatment two vials with 1000 NT 201 are reconstituted with 8.0 ml saline providing a solution with a concentration of 25 U/ml. For bilateral treatment 3 or 4 vials are reconstituted with 8.0 or 16.0 ml saline providing the same concentration. No further dilution is required.

For the initial treatment NT 201 is injected unilaterally into the muscles of the wrist/forearm, and mandatorily, into the elbow and shoulder muscles (table below, dosing scheme A). This dosing scheme is applicable for all patients with upper limb tremor of any intensity (mild—moderate-marked) and of any involvement of wrist/forearm/elbow/shoulder muscle groups.

Dosing scheme B can be used unilaterally or bilaterally initially by experienced injectors or after an initial injection to the patient using scheme A. For the dosing scheme B selection of tremulous muscles with flexible number of muscles in the forearm/wrist are to be decided by the investigator based upon clinical analysis and might be supported by EMG or any other technical supportive measures (e.g. kinematic analysis).

The total dose in the dosing scheme A is 140 U and the maximum permissible dose in dosing scheme B is 165 U per arm per patient. Forearm tremulous muscles are treated with a minimum of 4 and maximum of 7 muscles in the dosing scheme B. All 7 forearm/wrist muscles are treated in the dosing scheme A.

In bilateral treatment the total dose in the dosing scheme A is 280.0 U and in dosing scheme B is 330.0 U per patient. For the bilateral treatment the same rules of dosing apply as in unilateral treatment, just those are applied for both treated arms in parallel.

TABLE 1

| | Number | | Dosing scheme B |
|---|---|---|---|
| | of | Dosing scheme A | (flexible |
| | injection | (fixed forearm/wrist | forearm/wrist |
| Muscles | points | muscle & dose) | muscle & dose) |
| Forearm/wrist | | Total forearm | At least 4 of the |
| muscles | | dose: | following should |
| | | 40.0 U | be treated |
| | | | Total forearm |
| | | | dose range: |
| | | | 30.0-65.0 U |
| M.extensor carpi | 1 | 2.5 U | 2.5-5.0 U |
| ulnaris (ECU) | | | |
| M. extensor carpi | 1 | 2.5 U | 2.5-5.0 U |
| radialis (ECR) | | | |
| M. flexor carpi radialis | 1 | 10.0 U | 5.0-15.0 U |
| (FCR) | | | |
| M. flexor carpi ulnaris | 1 | 10.0 U | 5.0-15.0 U |
| (FCU) | | | |
| M. pronator teres (PT) | 1 | 10.0 U | 5.0-15.0 U |
| M. pronator quadratus | 1 | 2.5 U | 2.5-5.0 U |
| (PQ) | | | |
| M. supinator | 1 | 2.5 U | 2.5-5.0 U |
| Elbow muscles | | Elbow total dose: | Elbow total dose: |
| | | 40.0 U | 40.0 U |
| M. brachialis | 2 | 20.0 U | 20.0 U |
| M. triceps brachii | 2 | 20.0 U | 20.0 U |
| Shoulder muscles | | Shoulder total | Shoulder total |
| | | dose: | dose: |
| | | 60.0 U | 60.0 U |
| M. latissimus dorsi | 2 | 15.0 U | 15.0 U |
| M. pectoralis major | 2 | 15.0 U | 15.0 U |
| M. supraspinatus | 3 | 15.0 U | 15.0 U |
| M. infraspinatus | 3 | 15.0 U | 15.0 U |
| Total dose: | | 140.0 U | 130.0-165.0 U |

Injections can be guided by EMG, ultrasound or electrical stimulation of the muscles as to be decided on the discretion of the injector. A combination of those guidance techniques can also be used within one patient and also different techniques are possible for different muscles.

Example Treatment for Individual Patient

A male patient, having essential tremor in both of his arms, having a TETRAS score of 3 on the kinetic tremor of the upper limb (5 to less than 10 cm amplitude), receives a treatment with NT201 using the following fixed dosing scheme:

| | Number | |
|---|---|---|
| | of | Dosing scheme A |
| | injection | (fixed forearm/wrist |
| Muscles | points | muscle & dose) |
| Forearm/wrist | | Total forearm |
| muscles | | dose: |
| | | 40.0 U |
| M.extensor carpi | 1 | 2.5 U |
| ulnaris (ECU) | | |
| M. extensor carpi | 1 | 2.5 U |
| radialis (ECR) | | |
| M. flexor carpi radialis | 1 | 10.0 U |
| (FCR) | | |
| M. flexor carpi ulnaris | 1 | 10.0 U |
| (FCU) | | |
| M. pronator teres (PT) | 1 | 10.0 U |
| M. pronator quadratus | 1 | 2.5 U |
| (PQ) | | |
| M. supinator | 1 | 2.5 U |
| Elbow muscles | | Elbow total dose: |
| | | 40.0 U |
| M. brachialis | 2 | 20.0 U |
| M. triceps brachii | 2 | 20.0 U |
| Shoulder muscles | | Shoulder total |
| | | dose: |
| | | 60.0 U |
| M. latissimus dorsi | 2 | 15.0 U |
| M. pectoralis major | 2 | 15.0 U |
| M. supraspinatus | 3 | 15.0 U |
| M. infraspinatus | 3 | 15.0 U |
| Total dose: | | 140.0 U |

After 4 weeks the TETRAS score of the kinetic tremor of the right arm is decreased by 1 score points (−33%). After 8 weeks the TETRAS score is decreased by 0.5 score points (−17%) in comparison to just before the injection.

Patient with Semi-Flexible Dosing:

A male patient, having mainly a flexion/extension dominant wrist essential tremor with a TETRAS score of 2.5 score points according to the posture of the arm (tremor amplitude is between 3 to less than 5 cm) receives a treatment with NT201 using the following semi-flexible dosing scheme:

| | Number | Dosing scheme B |
|---|---|---|
| | of | (flexible |
| | injection | forearm/wrist |
| Muscles | points | muscle & dose) |
| Forearm/wrist | | At least 4 of the |
| muscles | | following should |
| | | be treated |
| | | Total forearm |
| | | dose range: |
| | | 30.0-65.0 U |
| M.extensor carpi | 1 | 2.5 U |
| ulnaris (ECU) | | |
| M. extensor carpi | 1 | 2.5 U |
| radialis (ECR) | | |
| M. flexor carpi radialis | 1 | 15.0 U |
| (FCR) | | |
| M. flexor carpi ulnaris | 1 | 15.0 U |
| (FCU) | | |
| M. pronator teres (PT) | 1 | 0 U |
| M. pronator quadratus | 1 | 0 U |
| (PQ) | | |
| M. supinator | 1 | 0 U |
| Elbow muscles | | Elbow total dose: |
| | | 40.0 U |
| M. brachialis | 2 | 20.0 U |
| M. triceps brachii | 2 | 20.0 U |
| Shoulder muscles | | Shoulder total |
| | | dose: |
| | | 60.0 U |
| M. latissimus dorsi | 2 | 15.0 U |
| M. pectoralis major | 2 | 15.0 U |
| M. supraspinatus | 3 | 15.0 U |
| M. infraspinatus | 3 | 15.0 U |
| Total dose: | | 135.0 U |

After 4 weeks the TERTAS score is decreased by 1 score points (−40%). After 8 weeks the TETRAS score is decreased by 0.5 score points (−25%) compared to the score just before the injection.

Patient with Semi-Flexible Dosing:

A female patient, having a flexion/extension dominant wrist essential tremor with a rotational component and with a TETRAS score of 3 score points according to the posture of the arm (tremor amplitude is between 5 to less than 10 cm) receives a treatment with NT201 using the following semi-flexible dosing scheme:

| | Number | Dosing scheme B |
|---|---|---|
| | of | (flexible |
| | injection | forearm/wrist |
| Muscles | points | muscle &dose) |
| Forearm/wrist | | 5 of the following |
| muscles | | should be treated |
| | | Total forearm |
| | | dose range: |
| | | 30.0-65.0 U |
| M. extensor carpi | 1 | 0 U |
| ulnaris (ECU) | | |
| M. extensor carpi | 1 | 2.5 U |
| radialis (ECR) | | |
| M. flexor carpi radialis | 1 | 15.0 U |
| (FCR) | | |
| M. flexor carpi ulnaris | 1 | 15.0 U |
| (FCU) | | |
| M. pronator teres (PT) | 1 | 5 U |
| M. pronator quadratus | 1 | 0 U |
| (PQ) | | |
| M. supinator | 1 | 2.5 U |
| Elbow muscles | | Elbow total dose: |
| | | 40.0 U |
| M. brachialis | 2 | 20.0 U |
| M. triceps brachii | 2 | 20.0 U |
| Shoulder muscles | | Shoulder total |
| | | dose: |
| | | 60.0 U |
| M. latissimus dorsi | 2 | 15.0 U |
| M. pectoralis major | 2 | 15.0 U |
| M. supraspinatus | 3 | 15.0 U |
| M. infraspinatus | 3 | 15.0 U |
| Total dose: | | 140.0 U |

After 4 weeks the TERTAS score is decreased by 1 score points (−33%). After 8 weeks the TETRAS score is still decreased by 1 score points (−33%) compared to the score just before the injection.

1. A botulinum neurotoxin adapted for treating a tremor of an upper limb comprising administering a botulinum neurotoxin to at least one muscle of a forearm/wrist, elbow and shoulder, wherein the botulinum neurotoxin is administered to at least one muscle of the forearm/wrist selected from the group of M.extensor carpi ulnaris (ECU), M.extensor carpi radialis (ECR), M.pronator quadratus (PQ) and M.supinator in a dosage in the range of 2 to 6 U and wherein the botulinum neurotoxin is administered to at least one muscle of the elbow in a dosage of about 20 U and to at least one muscle of the shoulder in a dosage of about 15 U.

2. The method of claim 20, wherein the botulinum neurotoxin is administered in addition to at least one muscle of the wrist/forearm selected from the group of M. flexor carpi radialis (FCR), M. flexor carpi ulnaris (FCU) and M. pronator teres (PT) in a dose in the range of 4 to 16 U per muscle.

3. The method of claim 20, wherein the botulinum neurotoxin is administered to at least one muscle of the forearm/wrist selected from the group of M. extensor carpi ulnaris (ECU), M. extensor carpi radialis (ECR), M. pronator quadratus (PQ) and M. supinator in a dosage of about 2.5 U.

4. The method according to claim 3, wherein the botulinum neurotoxin is administered in addition to at least one muscle of the wrist/forearm selected from the group of M. flexor carpi radialis (FCR), M. flexor carpi ulnaris (FCU) and M. pronator teres (PT) in a dose of about 10 U per muscle.

5. The method of claim 20, wherein the botulinum neurotoxin is not administered to the M. externsor digitorium communis.

6. The method of claim 20, wherein the total dose of the botulinum neurotoxin administered to the forearm/wrist muscles does not exceed 65 U.

7. The method of claim 20, wherein the botulinum neurotoxin is administered to at least one muscle of the elbow selected from the group of M. brachialis and M. triceps brachii.

8. The method of claim 20, wherein the botulinum neurotoxin is not administered to the M. biceps brachii.

9. The method of claim 20, wherein the total does of the botulinum neurotoxin administered to the muscles of the elbow does not exceed 40 U.

10. The method of claim 20, wherein the botulinum neurotoxin is administered to at least one muscle of the shoulder selected from the group of M. latissimus dorsi, M. pectoralis major, M. supraspinatus and M. infraspinatus.

11. The method of claim 20, wherein the botulinum neurotoxin is not administered to the deltoid and the teres major muscle.

12. The method of claim 20, wherein the total dose of the botulinum neurotoxin administered to muscles of the shoulder does not exceed 60 U.

13. The method of claim 20, wherein the total dose of the botulinum neurotoxin administered to the muscles of the forearm/wrist, elbow and shoulder does not exceed 165 U.

14. The method of claim 20, wherein the botulinum neurotoxin is administered to 4, 5, 6, or 7 muscles of the forearm/wrist, 2 muscles of the elbow and 4 muscles of the shoulder in a dose according to the scheme:

| | |
|---|---|
| M. extensor carpi ulnaris (ECU) | about 2.5 U to 5 U |
| M. extensor carpi radialis (ECR) | about 2.5 U to 5 U |
| M. flexor carpi radialis (FCR) | about 5 U to 15 U |
| M. flexor carpi ulnaris (FCU) | about 5 U to 15 U |
| M. pronator teres (PT) | about 5 U to 15 U |
| M. pronator quadratus (PQ) | about 2.5 U to 5 U |
| M. supinator | about 2.5 U to 5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U 55 |

15. The method of claim 20, wherein the botulinum neurotoxin is administered to 7 muscles of the forearm/wrist, 2 muscles of the elbow and 4 muscles of the shoulder in a dose according to the scheme:

| | |
|---|---|
| M. extensor carpi ulnaris (ECU) | about 2.5 U |
| M. extensor carpi radialis (ECR) | about 2.5 U |
| M. flexor carpi radialis (FCR) | about 10 U |
| M. flexor carpi ulnaris (FCU) | about 10 U |
| M. pronator teres (PT) | about 10 U |
| M. pronator quadratus (PQ) | about 2.5 U |
| M. supinator | about 2.5 U |
| M. brachialis | about 20 U |
| M. triceps brachii | about 20 U |
| M. latissimus dorsi | about 15 U |
| M. pectoralis major | about 15 U |
| M. supraspinatus | about 15 U |
| M. infraspinatus | about 15 U |

16. The method of claim 20, wherein said botulinum neurotoxin is a neurotoxic component of a botulinum neurotoxin complex, wherein said neurotoxic component is devoid of any other protein component of the Clostridium botulinum neurotoxin complex.

17. The method of claim 20, wherein said botulinum neurotoxin is one or more selected from the group of serotypes comprising A, B and E.

18. The method of claim 20, wherein the botulinum neurotoxin is administered together with at least one standard treatment selected from propranolol, primidone, any other antiepileptic or a calcium channel blocker, Deep Brain Stimulation (DBS), Magnetic Resonance guided High Frequency Ultrasound (MRgHiFUS), local electrical stimulation, biofeedback, kinematic assessment guided stimulation, anti-tremor appliances, anti-tremor smartphone apps or combinations thereof.

19. A pharmaceutical composition comprising a botulinum neurotoxin according to claim 1 for treating a tremor of the upper limb.

20. A method for treating a tremor of an upper limb wherein the method comprises administering a therapeutically effective amount of a botulinum neurotoxin according to claim 1.


SEQUENCE LISTING (14 sequences)

SEQ ID NO: 1 - DNA, 3891 bp, Clostridium botulinum
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3876 bp, Clostridium botulinum
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 4 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 3843 bp, Clostridium botulinum
atgccaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctcctcgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataaggtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgccataaagcaatagatggtagatcattatataataaaacattagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatataattaatgaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatacaataattaatagtataaaacaaaattctgggtggaaattatgtattaggaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttaaatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatattattaatgataattatatagataggtatatagcacctaaaagtaatatacttgtacttgttcagtatccagatagatctaaattatatactggaaatcctattactattaaatcagtatctgataagaatccttatagtagaattttaaatggagataatataatgtttcatatgttatataatagtgggaaatatatgataataagagatactgatacaatatatgcaatagaaggaagagagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagtaa

SEQ ID NO: 6 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 7 - DNA, 3858 bp, Clostridium botulinum
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcctagacctacttcaaagtatcaaagttattatgatcctagttatttatctactgatgagcaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattgtatggaataaatataccatctgataaaaggattcgtccacaagttagcgagggatttttttctcaagatggacccaacgtacaatttgaggaattatacacatttggaggatcagatgttgaaataatacctcaaattgaaagattacaattaagagaaaaagcattaggtcactataaagatatagcgaaaagacttaataatattaataaaactattccttctagttggagtagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaagcattatctacctgtatttgcaaatatattagatgataatatttatactataataaacggttttaatttaacaactaaaggttttaatatagaaaattcgggtcagaatatagaaaggaatcctgcactacaaaaacttagttcagaaagtgtagtagatttgtttacaaaagtatgtttaagattaacaagaaatagtagagatgattcaacatgtattcaagttaaaaataatacattaccttatgtagctgataaagatagcatttcacaagaaatatttgaaagtcaaattattacagatgagactaatgtagaaaattattcagataatttttcattagatgaatctattttagatgcaaaagtccctactaatcctgaagcagtagatccactgttacccaatgttaatatggaacctttaaatgttccaggtgaagaagaagtattttatgatgatattactaaagatgttgattatttaaactcttattattatttggaagcccaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaaaaaagatacattggataaaatatcagatgtatcagccataattccatatataggacctgccttaaatataggaaattcagcattaaggggaaactttaagcaagcatttgcaacagctggtgtagcttttttgttagaaggatttccagagtttacaatacctgcactcggtgtatttaccttttatagttctattcaagaaagagagaaaattattaaaactatagaaaattgtttagaacaaagagttaagagatggaaagattcatatcaatggatggtatcaaattggttgtcaagaattactactcgatttaatcatataagttatcaaatgtatgattctttgagttatcaggcagatgcaatcaaagctaaaatagatttagaatataaaaaatactcaggaagtgataaagaaaatataaaaagtcaagttgaaaatttaaaaaatagtttagatgtaaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatatgattaatgaatatttcaatagtattaatgattcaaaaattttgagcttacaaaataaaaaaaatactttgatggatacatcaggatataacgcagaagtgagagtagaaggcaatgttcagcttaatccaatatttccatttgactttaaattaggtagttcaggggatgatagaggtaaagttatagtaacccagaatgaaaatattgtatataatgctatgtatgaaagttttagtattagtttttggattaggataaataaatgggtaagtaatttacctggatatactataattgatagtgttaaaaataactcaggttggagtataggtattattagtaattttttagtgtttactttaaaacaaaatgaaaatagtgaacaagatataaactttagttatgatatatcaaagaatgctgcgggatataataaatggttttttgtaactattactaccaatatgatgggaaatatgatgatttatataaatggaaaattaatagatactataaaagttaaagagttaactggaattaattttagcaaaactataacatttcaaatgaataaaattccaaatactggcttaattacctcagattctgataacatcaatatgtggataagggatttttatatctttgctaaagaattagatgataaagatattaatatattatttaatagcttgcaatatactaatgttgtaaaagattattggggaaatgatttaagatatgataaagaatattacatgattaacgtaaattatatgaatagatatatgtctaaaaaaggcaatggaattgtttttaatacacgtaaaaataataatgacttcaatgaaggatataaaattataataaaaagaattagaggaaatacaaatgatactagagtacgaggagaaaatgtattatattttaatactacaattgataacaaacaatatagtttaggtatgtataaaccttctagaaatctagggactgatttagttccactaggtgcattggatcaaccaatggatgagatacgtaaatatggttcgtttataatacaaccatgcaatacttttgattactatgcatcacaattatttttgtcaagtaatgcaacaacaaataggcttggaatactatcaattggtagttatagtttcaaacttggagatgactattggtttaatcacgaatatttaattcctgttataaaaatagagcattatgcttcattattagaatcaacatcaactcattgggtttttgtacctgcaagtgaataa

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 9 - DNA, 3756 bp, Clostridium botulinum
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - DNA, 3843 bp, Clostridium botulinum
atgccagttgtaataaatagttttaattataatgaccctgttaatgatgagacaattttatacatgcagaaaccatatgaagaaagaagtagaaaatattataaagcttttgagattatgcctaatgtttggataatgcctgagagagatacaataggaactaagcctgatgagtttcaggtgccggattcattaaagaacggaagtagtgcttattatgatcctaattatttaaccactgatgctgaaaaagatagatatttaaaaacaatgataaaattatttaatagaattaatagtaatcctacagggaaagttttgttagaagaagtatcaaatgctagaccatatttaggagatgatgacacgctaattaatgaattccttccagttaatgtaactacaagtgttaatataaaattttcaactgatgttgaaagttcaataatatcgaatcttcttgtattgggagcaggacctgatatatttaaagcttactgtaccccccttgtaaggtttaataagtcagataaattaattgaaccaagtaatcatggttttggatcaattaatatcttgacattttcacctgagtatgaacatatttttaatgatattagtggagggaatcataatagtacagaatcatttattgcagatcctgcaatttcactagctcatgaattgatacatgcactacatggattatacggggctaaggcagttactcataaagagtctctagtagcagagcgaggacctcttatgatagccgaaaagcccataaggctagaagaatttttaacttttggaggtgaggatttaaatatcattcctagtgctatgaaggaaaaaatatataacgatcttttagctaactatgaaaaaatagctactagacttagagaagttaatacggctcctcctggatatgatattaatgaatataaagattattttcaatggaagtatggactagatagaaatgcagatggaagttatactgtgaatagaaataaatttaatgaaatttataaaaaattatatagctttacagagattgacttagcaaataaatttaaagtaaaatgtagaaatacttattttattaaatatggatttgtaaaagttccaaatttgttagatgatgatatttatactgtatcagaggggtttaatataggtaatttagcagtaaacaatcgcggacaaaatataaatttaaatcctaaaattattgattccattccagataaaggtttagtggaaaagattattaaattttgtaagagcattattcctagaaaaggtacgaagcagtcaccgtcactatgcattagagtaaataatagggagttattttttgtagcttcagaaagtagctataatgaaagtgatattaatacacctaaagaaattgacgatacaacaaatctaaataataattatagaaataatttagatgaagttattttagattataatagtgagacaatacctcaaatatcaaatcgaacattaaatacacttgtacaagacaatagttatgtgccaagatatgattctaatggaacaagtgaaatagaggaatatgatgttgttgactttaatgtatttttctatttacatgcacaaaaagtaccagaaggtgaaaccaatataagtttaacttcttcaattgatacagcattattagaagaatccaaagtatatacatttttttcttcagagtttatcgatactatcaataaacctgtaaatgcagcactatttatagattggataagcaaagtaataagagattttaccactgaagctacacaaaaaagtactgttgataagattgcagacatatctttaattgtaccctatgtaggtcttgctttgaatatagttattgaggcagaaaaaggaaattttgaggaggcatttgaattattaggagcgggtattttattagaatttgtgccagagcttacaattcctgtaattttagtgtttacgataaaatcctatatagattcatatgagaataaaaataaagcaattaaagcaataaataattcattaatcgaaagagaagcaaagtggaaagaaatatatagttggatagtatcaaattggcttactagaattaatacgcaatttaataaaagaaaagagcaaatgtatcaggctttacaaaatcaagtagatgcaataaaaacagcaatagaatataaatataataattatacttcagatgagaaaaatagacttgaatctaaatataatatcaataatatagaagaagaattgaataaaaaagtttctttagcaatgaaaaatatagaaagatttatgacagaaagttctatatcttatttaatgaaattaataaatgaagccgaagttggtaaattaaaagaatatgataaacatgttaagagcgatttattagactatattctctaccataaattaatcttaggagagcagacaaaggaattaattgatttggtgactagtactttgaatagtagtattccatttgaactttcttcatatactaatgataaaattctaattatatattttaatagattatataaaaaaattaaagatagttctattttagatatgcgatatgaaaataataaatttatagatatctctggatatggttcaaatataagcattaatggaaacgtatatatttattcaacaaatagaaatcaatttggaatatatagtggtaggcttagtgaagttaatatagctcaaaataatgatattatatacaatagtagatatcaaaattttagtattagtttctgggtaaccattcctaaacactacagacctatgaatcgtaatcgggaatacactataataaattgtatggggaataataattcgggatggaaaatatcacttagaactattagagattgtgaaataatttggactttacaagatacttccggaaataaggaaaaattaatttttaggtatgaagaacttgctagtatatctgattatataaataaatggatttttgtaactattactaataatagattaggcaattctagaatttacatcaatggaaatttaatagttgaaaaatcaatttcgaatttaggtgatattcatgttagtgataatatattatttaaaattgttggttgtgatgatgaaacgtatgttggtataagatattttaaagtttttaatacggaattagataaaacagaaattgagactttatatagtaatgagccagatccaagtatcttaaaagactattggggaaattatttgctatataataaaaaatattatttattcaatttactaagaaaagataagtatattactcggaattcaggcattttaaatattaatcaacaaagaggtgttactggaggcatatctgtttttttgaactataaattatatgaaggagtagaagttattataagaaaaaatgctcctatagatatatctaatacagataattttgttagaaaaaacgatctagcatacattaatgtagtagatcatggtgtagaatatcggttatatgctgatatatcaattacaaaatcagagaaaataataaaattaataagaacatctaatccaaacgatagcttaggtcaaattatagttatggattcaataggaaataattgcacaatgaattttcaaaacaatgatgggagcaatataggattactaggttttcattcagatgatttggttgctagtagttggtattataaccatatacgaagaaacactagcagtaatggatgcttttggagttttatttctaaagagcatggttggaaagaataa

SEQ ID NO: 12 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 13 - DNA, 3894 bp, Clostridium botulinum
  misc_feature (20)..(20): n is a, c, g, or t
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 14 - protein, 1297 aa, Clostridium botulinum
  MISC_FEATURE (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE